(19) United States
(12) Patent Application Publication
(10) Pub. No.: US 2016/0302723 A1
(43) Pub. Date: Oct. 20, 2016

(54) CROSS-LINKED PEG POLYMER COATING FOR IMPROVING BIOCOMPATIBILITY OF MEDICAL DEVICES

(71) Applicant: MEDICAL SURFACE INC, Natick, MA (US)

(72) Inventor: Xiaoxi Kevin Chen, Natick, MA (US)

(21) Appl. No.: 15/100,439

(22) PCT Filed: Dec. 4, 2014
(86) PCT No.: PCT/US14/68518

§ 371 Date: May 31, 2016

Related U.S. Application Data
(60) Provisional application No. 61/911,879, filed on Dec. 4, 2013.

(51) Int. Cl. A61B 5/00; A61B 5/1486; B05D 1/00; A61B 5/145; A61M 1/16

(52) U.S. Cl. CPC A61B 5/686; A61B 5/6801; A61B 5/14532; A61M 1/16; B05D 1/62; A61B 5/1486; A61M 2205/0238; A61B 2562/18

(57) ABSTRACT

The present invention relates to a cross-linked PEG polymer coating that is hydrophilic, lubricious, and resistant to adsorption of biological matters including proteins and cells. The coating is created using plasma glow discharge polymerization of organic compounds with a formula R(OCH₂CH₂)ₙOH, where R is an alkane group with 1-4 carbon atoms and n=1-6.

### CROSS-REFERENCE TO RELATED APPLICATION

This application claims priority of U.S. Provisional Patent Application No. 61/911,879, filed Dec. 4, 2013, the entire contents of which are incorporated by reference herein.

### FIELD OF THE INVENTION

The present invention discloses methods for producing a cross-linked PEG polymer coating using plasma glow discharge polymerization of organic compounds with a formula R(OCH₂CH₂)ₙOH, where R is an alkane group with 1-4 carbon atoms and n=1-6. Advantageously, such methods produce a cross-linked PEG polymer coating that is covalently attached to the substrate surface. The degree of cross-linking and thickness of the polymer coating can be controlled by the plasma glow discharge polymerization process parameters and the thickness can range from nanometers to micrometers. The cross-linked PEG polymer coating can be formed on various materials including those used in medical catheters, implants, sensors and contact lenses. Advantageously, such methods impart hydrophilic, lubricious, non-fouling and biocompatible properties to medical devices.

### BACKGROUND OF THE INVENTION

Biofouling, which is the accumulation of biological matter at surfaces, happens in virtually any environment in which natural and man-made materials are used. One example of surfaces prone to biofouling is related to medical devices used in human body. Components of biofluids such as proteins, cells and pathogens have a propensity to strongly adhere to surfaces, altering performance with potentially hazardous outcomes. Urinary tract infections resulting from microbial colonization of catheters represents the most common hospital-acquired infection. Implantable medical devices are also susceptible to microbially influenced corrosion (MIC) leading to the need for replacement surgeries with increased risk of infection.

Efforts to create reliable, long-term implantable biosensors such as glucose sensor have been impeded by the effects of the foreign body response (FBR) that introduces delayed response times as well as unpredictable sensor performance. FBR happens when almost any material is inserted into tissues, starting with the creation of a wound and the wound healing cascade. Instantaneously, proteins adhere to the biomaterial surface, which is the common initial phase of a biofouling process. The initial protein adsorption is an integral part of the overall FBR as the ensuing interface promotes the adhesion of inflammatory cells. Later the inflammatory cells deposit a dense collagenous capsule that blocks mass transport and/or electric communication between the implant and the body. This has been a major challenge for continuous glucose monitoring (CGM) devices implanted in subcutaneous tissues. The collagen encapsulation lacks the microvasculature of native tissue. As blood vessels are the primary source of glucose, such encapsulation hinders accurate measurements of blood glucose.

A common strategy for limiting biofouling of surfaces and improving biosensor performance is to graft an anti-fouling polymer onto a surface. One of the most extensively studied anti-fouling polymers is poly(ethylene glycol) (PEG), a water soluble polymer with low toxicity and extensive history of use in medicine and drug delivery. PEG can be grafted onto surfaces with appropriate chemical derivatization to reduce the nonspecific adsorption of proteins, cells and bacteria. Although the thermodynamic and molecular mechanisms for the protein and cell resistance of surface immobilized PEG are not completely understood, numerous studies have determined that steric hindrance effects, chain length, grafting density, chain conformation, and hydrophilic property of the grafted polymer play important roles in resisting protein adhesion. A highly hydrated layer of PEG chains is compressed (mobility reduced) when biomolecules or cells approach the surface, leading to a repulsive osmotic force that prevent the adhesion of the biomolecules/cells. Therefore, PEG coating has been used to prevent protein adsorption, cell attachment and bacterial adhesion on the surface of medical devices.

Methods of PEG polymer coating include passive or covalent attachment of the PEG polymer on the surface. In the passive coating methods, PEG polymers are conjugated to proteins or other polymers that facilitate the adsorption onto biomaterial surfaces. Passive coating is performed by contacting the substrate surface with coating solutions, using processes such as spray coating or dip coating. The passive coating methods have the advantage of being easy to manufacture, but has the disadvantage of being less durable. The coated layer is prone to dissociation in the in vivo environment.

In the prior art covalent coating methods, PEG polymers bearing chemically reactive group are synthesized and covalently attached to the chemically reactive groups on the surface. This requires the presence of chemically reactive groups (such as amine or carboxyl functional groups) on the surface for attachment. As these groups are not present in most common biomaterials, an additional surface “priming” step is performed to impart functional groups on the surface by surface modification methods such as photochemistry, plasma treatment or plasma polymerization. Therefore the prior art covalent coating process contains several steps that induce higher manufacturing cost. In some methods, organic solvents or toxic chemicals are used in the reactions, making the methods unsuitable for some biomaterials.

In the prior art covalent coating methods, there is only a single layer of the PEG molecule attached to the surface. The thickness of the PEG layer is determined by the size of the PEG molecule and is usually limited to nanometer scale. The thin layer of PEG coating is susceptible to pin holes due to incomplete coatings. The pin holes can provide binding sites for biomolecules and micro-organisms and therefore reduce the antifouling performance. Since there is only one covalent attachment point per PEG molecule, the breakage of the attachment point (e.g, by hydrolysis or reduction) will dissociate the PEG molecule and expose original surface, thus forming a pin hole. Therefore the durability of the prior art covalent PEG coating is limited due to the single layer of PEG molecules and the single point attachment for each PEG molecule.

### SUMMARY OF THE INVENTION

A method is disclosed herein for creating a cross-linked polymer coating with poly(ethylene glycol) functional groups on surfaces using plasma glow discharge polymerization of organic compounds with a formula R(OCH₂CH₂)ₙOH, where R is an alkane group with 1-4 carbon atoms and n=1-6.

One advantage of the disclosed method is that the thickness and degree of cross-linking of the PEG polymer coating are customizable. Since the polymer is formed by covalently attaching layers after layers of monomers on the surface during the plasma polymerization process, the thickness of the film can increase indefinitely as the processing time increases. The degree of cross-linking can be controlled by the power of plasma glow discharge. Whereas in the prior art covalent PEG coating methods, each PEG molecule is covalently attached to the surface through a single point attachment; there is only a single layer of PEG molecules and therefore the thickness of the coating is limited by the size of the PEG molecule used for coating.

Another advantage of the disclosed method is that the cross-linked PEG coating impart hydrophilic, lubricious, non-fouling, and biocompatible properties to the coated substrates. Compare to prior art methods, this coating process eliminates pin-holes, and produces a cross-linked PEG polymer coating that is highly durable and resistance to adsorption of biological matters including proteins and cells. The coating can be formed on various materials including those used in medical catheters, implants, sensors and contact lenses.

A further advantage of the disclosed method is that the cross-linked PEG coating is permeable to small molecules such as glucose. In order for a coating to work well with an implanted or wearable biosensor such as a glucose monitoring device, not only it is important that the coating improves the biocompatibility of the device, but also it is important that the coating does not restrict the transport of analyte (such as blood glucose) from outside of the sensor to the detection component (such as the enzyme layer or the electrode layer) inside the sensor. If the coating of the glucose sensors restricts analyte transport, accumulation of glucose outside the sensor may occur, resulting in a boundary layer. Analyte concentrations inside the sensor will be substantially lower, due to analyte consumption by the sensor and the retardation of analyte diffusion through the coating. This will result in sensor inaccuracy. Since the cross-linked PEG coating is permeable to small molecules such as glucose, the coating will not retard analyte transport and can be used for the surface of biosensors where small molecule analytes are required to diffuse into the sensor for detection.

An additional advantage of the disclosed method is that the cross-linked PEG coating process is solvent-free and is compatible with biosensor enzymes and proteins; i.e., the coating process does not affect the function of enzymes and proteins already immobilized on the biosensor surface.

These and other features of the invention will be better understood through a study of the following detailed description and accompanying drawings.

### BRIEF DESCRIPTION OF THE FIGURES

FIG. 1 is a drawing representing a substrate coated with a cross-linked PEG polymer.

FIG. 2 is a drawing representing a dialysis membrane with both sides coated with a cross-linked PEG polymer.

FIG. 3 is a chart showing the thickness of the cross-linked PEG polymer coating as a function of coating time. The film thickness was measured by quartz crystal microbalance (QCM).

FIG. 4 is a chart comparing the adsorption of Immunoglobin G-horseradish peroxide conjugate (IgG-HRP) on three different surfaces: the first surface was uncoated, the second surface was coated with a single layer of PEG (prior art) and the third surface was coated with cross-linked PEG polymer (subject invention). The amount of IgG-HRP conjugate adsorbed on the surfaces was quantified by the HRP catalyzed oxidation of TMB (3,3′, 5,5′ tetramethylbenzidine), which changes color upon oxidation.

FIG. 5 is a chart comparing the adsorption of human fibronectin (HFN) on two different surfaces: one surface was uncoated and the other surface was coated with cross-linked PEG polymer (subject invention). The amount of HFN adsorbed on the surfaces was quantified by incubation with an anti-HFN IgG-HRP solution followed by the HRP catalyzed oxidation of TMB.

FIG. 6 is a chart comparing the attachment of cells on two different surfaces: one surface was uncoated and the other surface was coated with cross-linked PEG polymer (subject invention). Three cell types were tested: an immortalized epithelial cell line, an immortalized fibroblast cell line and a fibrosarcoma cancer cell line.

FIG. 7 is a chart comparing the static and kinetic coefficient of friction of two silicone substrates. One silicone substrate was uncoated and the other silicone substrate was coated with cross-linked PEG polymer (subject invention). The static and kinetic coefficients of friction were tested following testing method ASTM D1894.

FIG. 8 is a chart comparing the permeability of glucose through two dialysis membranes. One membrane was uncoated and the other membrane was coated with cross-linked PEG polymer (subject invention). The amount of glucose permeated through membrane was quantitated using a glucose assay kit.

FIG. 9 is a chart comparing the response of two glucose sensors (containing glucose oxidase) to different levels of glucose in the test solution. One sensor was uncoated and the other sensor was coated with cross-linked PEG polymer (subject invention).

### DETAILED DESCRIPTION OF THE INVENTION

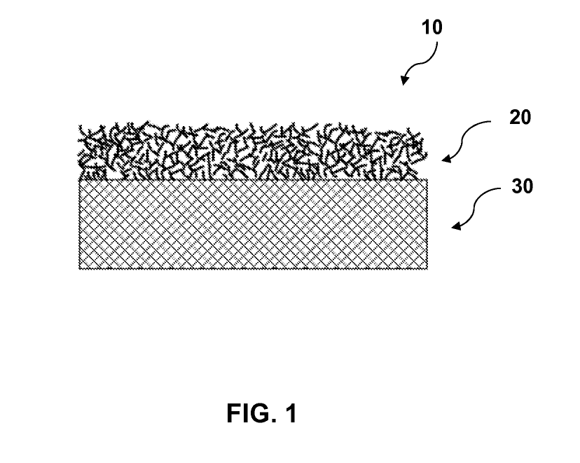

With reference to FIG. 1, a device 10 is depicted of comprising a substrate 30 and a coating composition 20. The coating composition 20 is produced by, i) providing a monomer source comprising one or more organic compounds, wherein at least one organic compound is R(OCH₂CH₂)ₙOH, where R is an alkane group with 1-4 carbon atoms and n=1-6; ii) creating a plasma of the monomer source; and iii) contacting at least a portion of a substrate 30 with the plasma to create a plasma polymer coated surface.

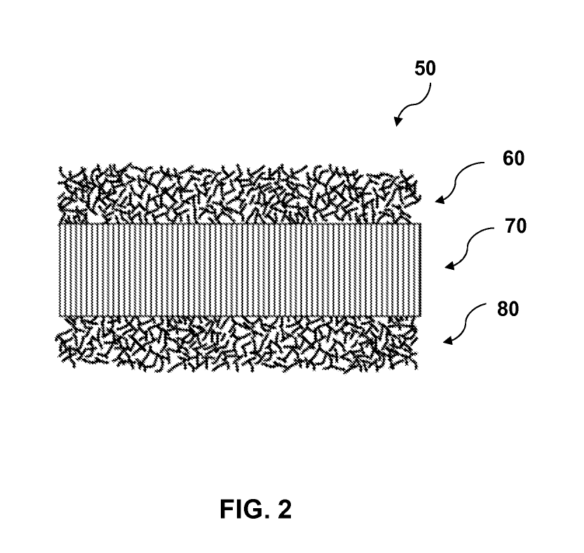

With reference to FIG. 2, a device 50 is depicted of comprising a dialysis membrane 70, a coating composition 60 on one side of the membrane, and a coating composition 80 on the other side of the membrane. The coating compositions 60 and 80 can be the same or different. The coating compositions 60 and/or 80 is produced by, i) providing a monomer source comprising one or more organic compounds, wherein at least one organic compound is R(OCH₂CH₂)ₙOH, where R is an alkane group with 1-4 carbon atoms and n=1-6; ii) creating a plasma of the monomer source; and iii) contacting at least a portion of the dialysis membrane 70 with the plasma to create a plasma polymer coated surface.

Any known technique can be used to generate plasma. The plasma may be generated using AC or DC power, radio-frequency (RF) power or micro-wave frequency power. Preferably, the plasma system is driven by a single radio-frequency (RF) power supply; typically at 13.56 MHz. The plasma system can either be capacitively coupled plasma, or inductively coupled plasma.

The substrate may be made of any materials, including polymers, glass, metal and silicon. Examples of polymers include polystyrene, polypropylene, polyethylene, polyester, silicone, polyurethane, ABS, PVC, polytetrafluoroethylene, polyvinylidene, and mixtures thereof. In one example, the substrates are continuous glucose monitoring devices with a polymer outer membrane. In another example, the substrates are coronary stent made of metal. In another example, the substrates are urinary catheters made of silicone material. In another example, the substrates are contact lenses made of silicone material.

In a preferred embodiment, the monomer used is Tri(ethylene glycol) monoethyl ether (CH₃CH₂(OCH₂CH₂)₃OH) or Tri(ethylene glycol) monomethyl ether (CH₃ (OCH₂CH₂)₃OH). Chemical compounds with similar molecular structure, specifically those containing saturated hydrocarbons on one end and ethylene glycol oligomers on the other end, can also be used. In the plasma state, the saturated hydrocarbons are ionized and can react with the surface of the substrate, forming a covalently bound thin film containing ethylene glycol oligomers. The substrates coated with this thin film of ethylene glycol oligomers obtain the ability to resist protein binding and cell attachment. The treated surfaces become non-fouling and anti-microbial due to the ability to resist binding/attachment of macromolecules and micro-organisms.

### EXAMPLES

### Example A

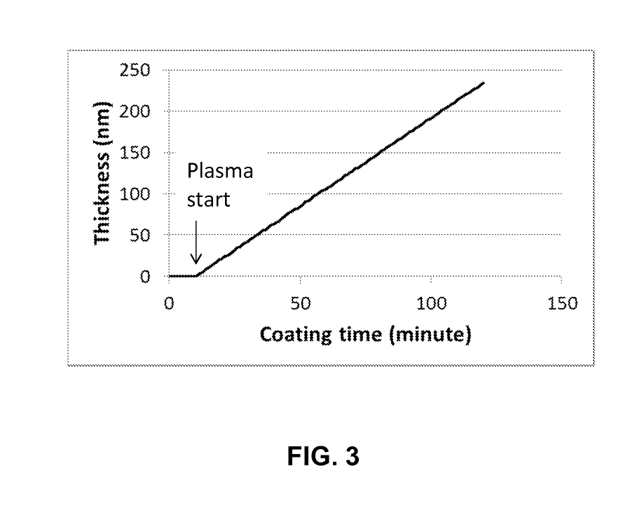

A quartz crystal micro-balance (QCM) gold plated crystal was coated with the cross-linked PEG coated surface of subject invention using plasma glow discharge polymerization of tri(ethylene glycol) monoethyl ether. The thickness of the coating was monitored by the frequency of the crystal. A plot of the thin film thickness versus time is shown in FIG. 3. The thickness increases linearly with time at a rate of approximately 2 nm per minute.

### Example B

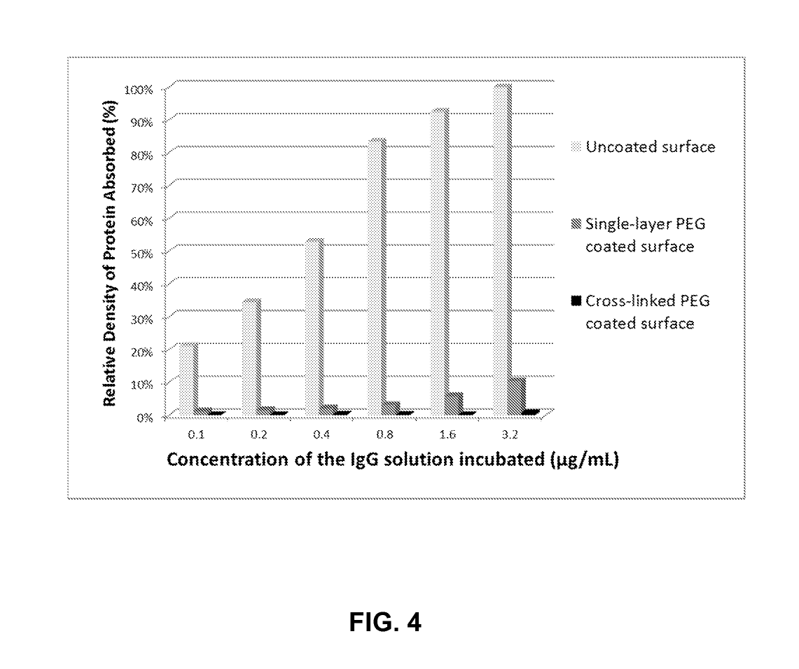

The cross-linked PEG coated surface of subject invention was compared with prior art single layer PEG coated surface and uncoated surface for IgG-HRP (Immunoglobin G-horseradish peroxide conjugate) binding. The cross-linked PEG coating was created using the subject invention plasma glow discharge polymerization method with tri(ethylene glycol) monoethyl ether as the monomer source. The traditional single layer PEG coating was created by first coating the surface with an acrylic acid plasma polymer, followed by reacting a high molecular weight PEG-amine molecule (MW 1000) with the carboxyl groups on the surface using well-established carbodiimide chemistry. The surfaces were exposed to increasing concentrations of IgG-HRP in PBS for 24 hours, followed by rinsing with PBS. The surfaces were then brought into contact with TMB (3,3′, 5,5′ tetramethylbenzidine) solution for 10 minutes followed by adding 1N HCl to stop the reaction. The amount of IgG-HRP bound on the surfaces was quantified by the intensity of the color (detected at 450 nm) produced by the oxidized TMB. As can be seen in FIG. 4, at all concentrations of IgG-HRP tested (up to 3.2 μg/ml), the cross-linked PEG coated surfaces showed no significant protein binding. The uncoated surface showed significant and increasing amounts of protein bound to the surface as expected. The traditional covalent PEG coated surfaces showed reduced but still detectable protein binding.

### Example C

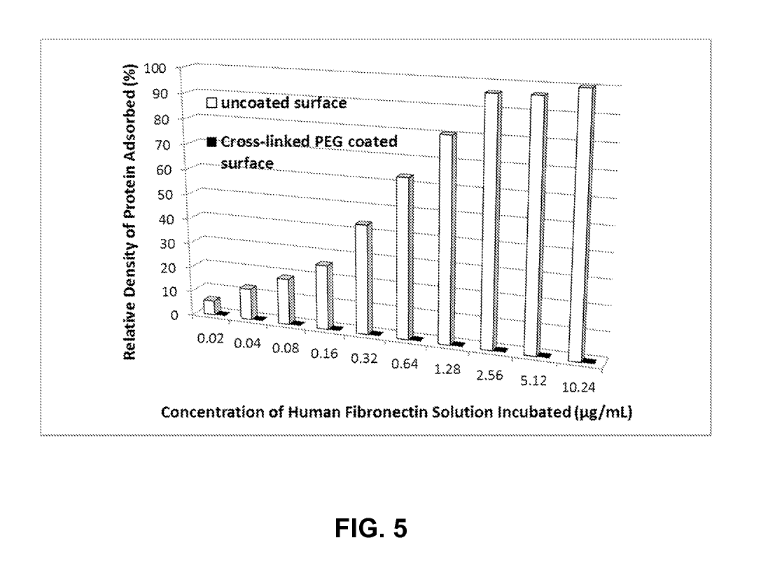

The cross-linked PEG coated surface of subject invention was compared with uncoated surface for human fibronectin (HFN) binding. The cross-linked PEG coating was created using the subject invention plasma glow discharge polymerization method with tri(ethylene glycol) monoethyl ether as the monomer source. The surfaces were exposed to increasing concentrations of HFN in PBS for 24 hours, followed by rinsing with PBS. Next the surfaces were exposed to a 0.5 μg/mL anti-HFN-IgG-HRP solution in PBS containing 0.5% BSA for 2 hours to allow the anti-HFN-IgG-HRP binding to any HFN adsorbed on the surfaces. The surfaces were rinsed with PBS again to remove excess anti-HFN-IgG-HRP. The surfaces were then brought into contact with TMB solution for 10 minutes followed by adding 1N HCl to stop the reaction. The amount of HFN/anti-HFN-IgG-HRP complex bound on the surfaces was quantified by the intensity of the color (detected at 450 nm) produced by the oxidized TMB. As can be seen in FIG. 5, at all concentrations of HFN tested (up to 10.2 μg/ml), the cross-linked PEG coated surfaces showed no significant protein binding. The uncoated surface showed significant and increasing amounts of protein bound to the surface as expected.

### Example D

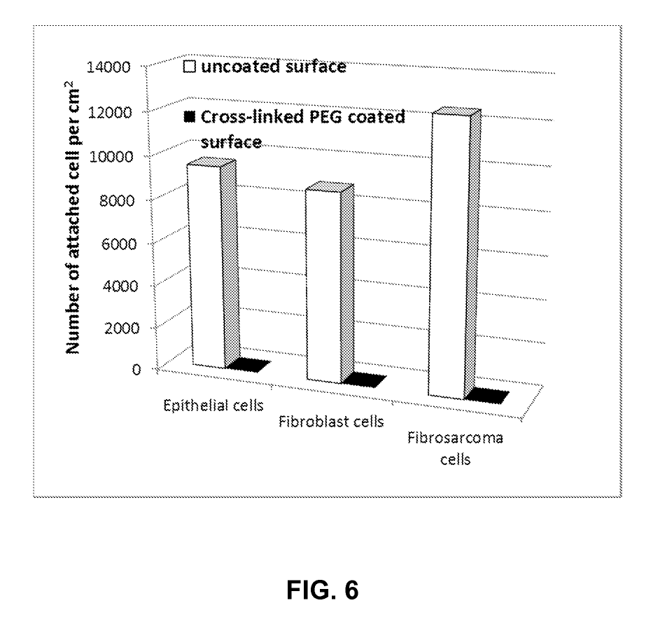

The cross-linked PEG coated surface of subject invention was compared with uncoated surface for cell attachment using several cell lines. The cross-linked PEG coating was created using the subject invention plasma glow discharge polymerization method with tri(ethylene glycol) monoethyl ether as the monomer source. The surfaces were incubated with 3 adherent cell lines: human epithelial cell LNCap, human fibroblast MRC5, and human fibrosarcoma cancer cell line HT1080. As can be seen in FIG. 6, while the cells adhered and proliferated on the uncoated surface, no cells were observed to adhere on the highly cross-linked PEG coated surface throughout the entire culture duration.

### Example E

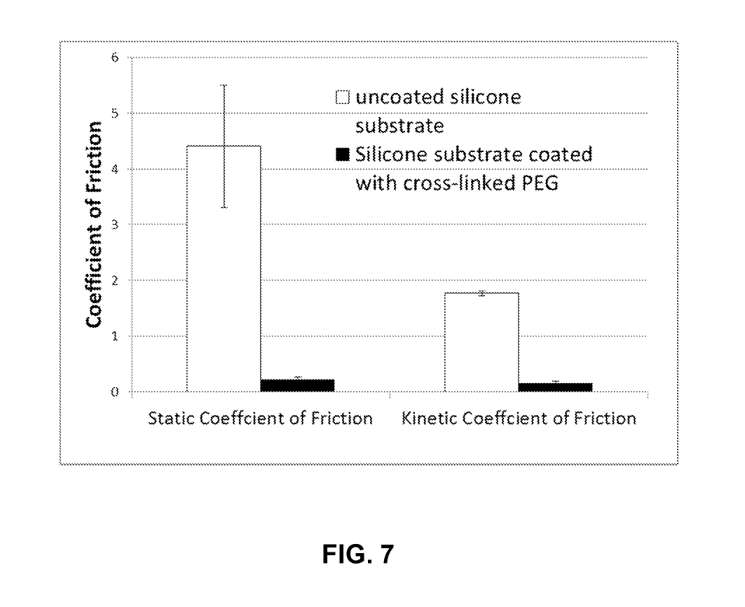

The cross-linked PEG coated silicone substrate of subject invention was compared with uncoated silicone substrate for wettability and lubricity. The cross-linked PEG coating was created using the subject invention plasma glow discharge polymerization method with tri(ethylene glycol) monoethyl ether as the monomer source. Wettability of the silicone substrate was measured by static contact angle of water droplets. The uncoated silicone substrate has a static contact angle of more than 100 degree, while the coated silicone substrate has a static contact angle of less than 60 degree. Lubricity of the silicone substrate was measured by static and kinetic coefficient of friction per testing method ASTM D1894. As can be seen in FIG. 7, more than 10-fold reduction of the coefficient of friction was observed for coated silicone substrate compared to uncoated silicone substrate.

### Example F

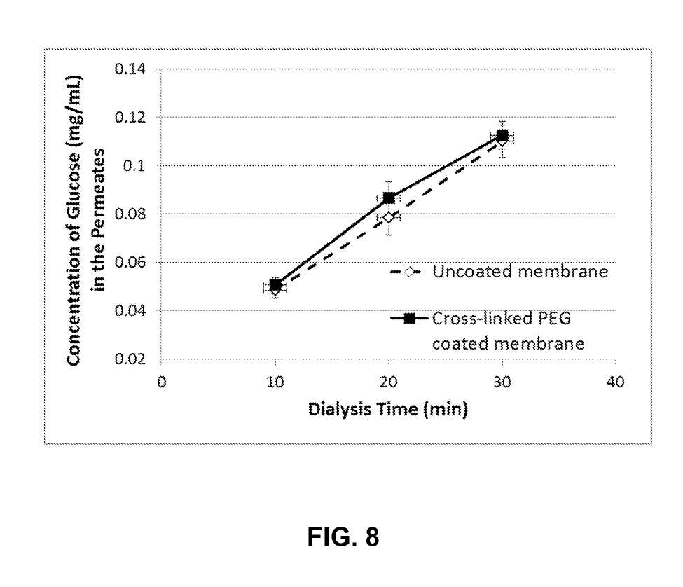

In order to investigate glucose permeation through the cross-linked PEG coating of subject invention, some dialysis membranes (3.5 kD MWCO) were coated with cross-linked PEG using the same coating parameters used in the protein and cell binding experiments shown in Examples B-D. Permeability of glucose across the coated and uncoated dialysis membrane was compared. The amount of glucose permeated through membrane was quantitated using a glucose assay kit (Sigma GAHK20). As can be seen in FIG. 8, there is no significant difference between the permeability of glucose through dialysis membrane coated with cross-linked PEG compared to uncoated dialysis membrane. Therefore, the cross-linked PEG coating does not retard glucose transport.

### Example G

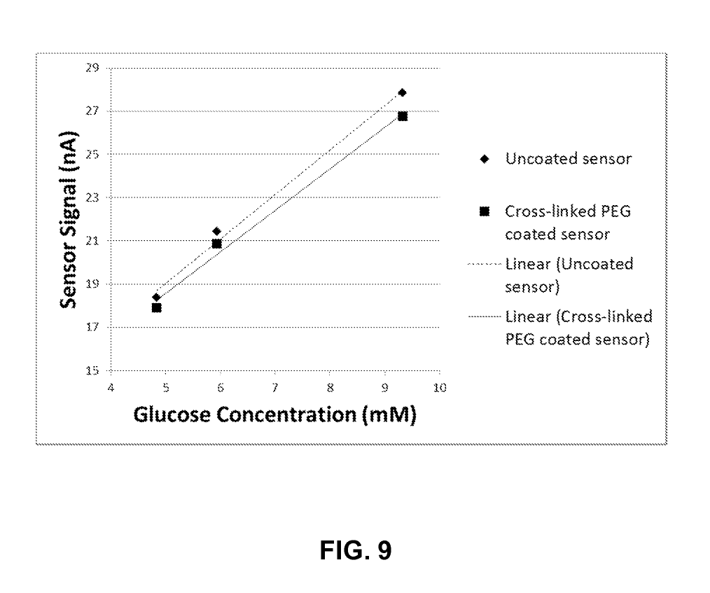

In order to investigate the impact of cross-linked PEG coating of subject invention on the enzymes immobilized on biosensor surface, glucose sensors with glucose oxidase immobilized on electrode surface were coated with cross-linked PEG using the same coating parameters used in the protein and cell binding experiments shown in Examples B-D. The coated and uncoated glucose sensors were exposed to test solutions with different glucose concentrations and the electrical currents generated by the glucose oxidase coated electrode were measured. As can be seen in FIG. 9, there is no significant difference between the uncoated sensor and the sensor coated with cross-linked PEG. Therefore, the cross-linked PEG coating does not affect the function of glucose oxidase on the electrode.

As will be appreciated by those skilled in the art, the subject invention can be used to prepare surfaces to improve wettability, lubricity, and resistance to binding of proteins and cells, and subsequently become biocompatible and non-fouling. Non-fouling surfaces obtained by the subject invention can be used to minimize foreign body reaction and prevent biofilm formation in medical devices and medical implants. By way of non-limiting example, the subject invention can be used to prepare surfaces of glucose monitoring sensors. By minimizing foreign body reaction, the non-fouling coating of subject invention can improve the performance of the implanted glucose sensor and prolong the sensor life. The subject invention can also be used to prepare surfaces of other medical devices such as artificial pancreas, hemodialysis devices, contact lenses, central venous catheters and needleless connectors, endotracheal tubes, intrauterine devices, mechanical heart valves, pacemakers, peritoneal dialysis catheters, prosthetic joints, tympanostomy tubes, urinary catheters, and voice prostheses.

What is claimed is:

1. A device comprising a substrate and a coating composition, said coating composition being produced by, i) providing a monomer source comprising one or more chemical compounds, wherein at least one chemical compound is R(OCH₂CH₂)ₙOH, where R is an alkane group with 1-4 carbon atoms and n=1-6; ii) creating a plasma of the monomer source; and iii) contacting at least a portion of a substrate with the plasma to provide a plasma polymer coated surface wherein the plasma polymer coated surface is hydrophilic, lubricious, and has the characteristics of resisting protein adsorption and cell adhesion.

2. A device of claim 1, wherein the substrate is a medical implant or a wearable medical device.

3. A device of claim 1, wherein the substrate is an implantable or wearable biosensor.

4. A device of claim 1, wherein the substrate is a glucose monitoring device.

5. A device of claim 1, wherein the substrate is a dialysis membrane.

6. A device of claim 1, wherein the substrate is a hemodialysis device.

7. A device of claim 1, wherein the substrate is a contact lens.

8. The method of claim 1, wherein said chemical compound is selected from one or a mixture of the following: Tri(ethylene glycol) monoethyl ether (CH₃CH₂(OCH₂CH₂)₃OH) and Tri(ethylene glycol) monomethyl ether (CH₃(OCH₂CH₂)₃OH).
